(19) United States
(12) Patent Application Publication
(10) Pub. No.: US 2025/0140376 A1
(43) Pub. Date: May 1, 2025

(54) Nutrient Infusion Control Method, Apparatus, Device, and Storage Medium

(71) Applicant: Medcaptain Medical Technology Co., Ltd., Shenzhen (CN)

(72) Inventors: Guangyu LIU, Shenzhen (CN); Xiaocheng TANG, Shenzhen (CN); Ling HU, Shenzhen (CN); Zhiwu DENG, Shenzhen (CN); Yazhou TANG, Shenzhen (CN)

(73) Assignee: Medcaptain Medical Technology Co., Ltd., Shenzhen (CN)

(21) Appl. No.: 18/902,697

(22) Filed: Sep. 30, 2024

(30) Foreign Application Priority Data
Oct. 26, 2023 (CN) 202311404897.X

(51) Int. Cl. G16H 20/60; A61M 1/00; G16H 10/60; G16H 40/63

(52) U.S. Cl. CPC G16H 20/60; A61M 1/00; G16H 10/60; G16H 40/63; A61M 2202/0007; A61M 2205/3303; A61M 2205/3331; A61M 2205/3379; A61M 2205/502; A61M 2205/52; A61M 2230/005; A61M 2230/04; A61M 2230/201; A61M 2230/30; A61M 2230/40

(57) ABSTRACT

The invention provides a nutrient infusion control method, apparatus, device, and storage medium. The method includes: acquiring at least one of nutrient infusion demand information, subject information, and vital sign status parameter of a current subject; determining a first nutrient infusion parameter according to subject the acquired information or parameter; and performing a nutrient infusion according to the first nutrient infusion parameter. The first nutrient infusion parameter is quickly and automatically determined directly according to at least one of the nutrient infusion demand information, the subject information, and the vital sign status parameter. This approach reduces reliance on nutritionists, enhances data accuracy, and improves the precision of nutrient infusion control. Furthermore, this method allows for rapid and accurate determination of the first nutrient infusion parameters, and thus controlling the nutrient infusion device with higher practicality.

### REFERENCE TO RELATED APPLICATIONS

The invention claims priority of Chinese Patent Application No. 202311404897.X, entitled “Nutrient Infusion Control Method, Apparatus, Device and Storage Medium” filed with the China National Intellectual Property Administration on Oct. 26, 2023, which is incorporated herein by reference in its entirety.

### TECHNICAL FIELD

This invention generally relates to the technical field of medical devices, and more particularly, to a nutrient infusion control method, apparatus, device and storage medium.

### BACKGROUND ART

Nutrient feeding is often administered to patients during their treatment and recovery when their own nutrient intake is insufficient to support normal body nutrient requirements. Through nutrient feeding, the patient can be provided with sufficient nutrients to support the patient's life activities and help the patient to recover.

In the prior art, a professional nutritionist usually formulates a personalized nutrient feeding plan according to a doctor's order and the patient's own basic information, vital signs status information, and other information to provide appropriate nutrient support, and periodically checks the performance of the vital signs of the subject, that is, the person who needs feeding, such as a patient or other person, during the nutrient feeding process to adjust the nutrient infusion parameters.

However, this method heavily relies on the experience and expertise of nutritionists, leading to potential inaccuracies in data. It is also not suitable for home environments, as its automation and practicality are limited, thereby failing to provide optimal nutrient infusion for the subject.

### SUMMARY OF THE INVENTION

The invention provides a nutrient infusion control method, apparatus, device and storage medium for solving the problem of how to improve the accuracy and efficiency of parameter determination.

In a first aspect, the invention provides a nutrient infusion control method, comprising:
acquiring at least one of: nutrient infusion demand information, subject information, and a vital sign status parameter of a current subject;
determining a first nutrient infusion parameter according to at least one of the nutrient infusion demand information, the subject information, and the vital sign status parameter; and
performing a nutrient infusion according to the first nutrient infusion parameter.

In one embodiment, the nutrient infusion demand information comprises one or more of the following: a nutrient solution type, a feeding mode, a feeding rate, a feeding amount, and a feeding time;
the subject information comprises one or more of the following: weight, weight change, age, and caloric consumption;
the vital sign status parameter comprises one or more of the following: an end-expiratory carbon dioxide indicator, a hemodynamic indicator, a blood glucose indicator, a heart rate, a blood pressure, an electrocardiogram, a body temperature, a blood oxygen saturation, and a pulse;
the first nutrient infusion parameter comprises one or more of the following: a type of nutrient solution, an infusion mode, an infusion rate, and an infusion volume.

In one embodiment, the method further comprises:
displaying an interface for adjusting nutrient infusion parameters;
receiving input from the user in the interface for adjusting nutrient infusion parameters to determine a second nutrient infusion parameter;
adjusting the first nutrient infusion parameter according to the second nutrient infusion parameter input by the user.

In one embodiment, before the step of performing the nutrient infusion according to the first nutrient infusion parameter, the method further comprises:
displaying the first nutrient infusion parameter;
receiving a confirmation operation from the user to adopt the first nutrient infusion parameter.

In one embodiment, the step of determining the first nutrient infusion parameter according to at least one of the nutrient infusion demand information, the subject information, and the vital sign status parameter, comprises:
inputting at least one of the nutrient infusion demand information, the subject information, and the vital sign status parameter into a predetermined data analysis model, and determining an infusion output strategy from the predetermined data analysis model as the first nutrient infusion parameter.

In one embodiment, the step of determining the first nutrient infusion parameter according to at least one of the nutrient infusion demand information, the subject information, and the vital sign status parameter comprises:
retrieving historical feeding parameters of the current subject;
comparing and matching the nutrient infusion demand information, the subject information, and the vital sign status parameter with the historical feeding parameters; and
determining the historical feeding parameter with the highest degree of matching as the first nutrient infusion parameter.

In one embodiment, the method further comprises:
acquiring the vital sign status parameter and an infusion parameter of the current subject during the nutrient infusion process; and
adjusting the first nutrient infusion parameter according to a preset parameter adjustment strategy when it is determined that at least one of the vital sign status parameter and the infusion parameter satisfies a preset parameter adjustment condition.

In one embodiment, the method further comprises:
recording details of parameter adjustment during the nutrient infusion process; wherein the details of parameter adjustment comprise a parameter adjustment time and/or values before and after the parameter adjustment.

In a second aspect, the invention provides a nutrient infusion control device, comprising:
an acquisition unit configured to acquire at least one of nutrient infusion demand information, subject information, and vital sign status parameter of a current subject;
a parameter determination unit configured to determine a first nutrient infusion parameter according to at least one of the nutrient infusion demand information, the subject information, and the vital sign status parameter; and
a control unit configured to perform a nutrient infusion according to the first nutrient infusion parameter.

In a third aspect, the invention provides a nutrient infusion device, wherein the nutrient infusion device is provided with an integrated monitoring module. The integrated monitoring module is configured to collect vital sign status parameter of the subject. The electronic device comprises a processor and a memory communicatively connected to the processor. The memory stores computer-executable instructions and the processor is configured to execute the computer-executable instructions stored in the memory to implement the method as previously described.

In one embodiment, the integrated monitoring module comprises one or more of the following modules: a respiratory parameter detection module, a hemodynamic detection module, and a blood glucose detection module. The respiratory parameter detection module is configured to collect end-expiratory carbon dioxide indicators. The hemodynamic detection module is configured to collect hemodynamic indicators, and the blood glucose detection module is configured to collect blood glucose indicators.

In one embodiment, the integrated monitoring module is set to be pluggable, that is, can be plugged into the nutrient infusion device.

In a fourth aspect, the invention provides a computer-readable storage medium, which has computer-executable instructions stored therein, and which are used for realizing the method as previously described when executed by a processor.

In a fifth aspect, the invention provides a computer program product comprising a computer program, which, when executed by a processor, is configured to implement a method as previously described.

The invention provides a nutrient infusion control method, apparatus, device, and storage medium, including: acquiring at least one of nutrient infusion demand information, subject information, and a vital sign status parameter of a current subject; determining a first nutrient infusion parameter according to at least one of the nutrient infusion demand information, the subject information, and the vital sign status parameter; and performing a nutrient infusion according to the first nutrient infusion parameter. In the invention, the first nutrient infusion parameter is quickly and automatically determined directly according to at least one of the nutrient infusion demand information, the subject information, and the vital sign status parameter. This approach reduces reliance on nutritionists, enhances data accuracy, and improves the precision of nutrient infusion control. Furthermore, this method is applicable to various practical scenarios, allowing for rapid and accurate determination of the first nutrient infusion parameters even in home environments, and thus controlling the nutrient infusion device with higher practicality.

### BRIEF DESCRIPTION OF THE DRAWINGS

The accompanying drawings herein, which are incorporated into and form a part of the specification, illustrate embodiments consistent with the invention and are used in conjunction with the specification to explain the principles of the invention.

FIG. 1 shows a schematic diagram of one application scenario of a nutrient infusion control method provided by embodiments of the invention.

FIG. 2 shows a schematic diagram of another application scenario of the nutrient infusion control method provided by embodiments of the invention.

FIG. 3 shows a schematic diagram of yet another application scenario of the nutrient infusion control method provided by embodiments of the invention.

FIG. 4 shows a structural schematic diagram of a nutrient infusion apparatus provided by embodiments of the invention.

FIG. 5 shows a flowchart of the nutrient infusion control method provided by embodiments of the invention.

FIG. 6 is a schematic diagram of an interface for displaying nutrient infusion parameters provided by embodiments of the invention.

FIG. 7 shows a schematic diagram of an interface for adjusting the nutrient infusion parameters provided by embodiments of the invention.

FIG. 8 shows a schematic diagram of another interface for adjusting nutrient infusion parameters provided by embodiments of the invention.

FIG. 9 shows a schematic flowchart of yet another nutrient infusion control method provided by embodiments of the invention.

FIG. 10 is a structural schematic diagram of a nutrient infusion control apparatus provided by embodiments of the invention.

FIG. 11 is a structural schematic diagram of another nutrient infusion control apparatus provided by embodiments of the invention.

FIG. 12 is a structural schematic diagram of a nutrient infusion device provided by embodiments of the invention.

By means of the above accompanying drawings, various embodiments of the invention are shown, and will be described in more detail below. The accompanying drawings and textual descriptions are not intended to limit the scope of the invention idea in any way, but rather to illustrate the concepts of the invention for those skilled in the art by reference to particular embodiments.

### DETAILED DESCRIPTION

Examples of embodiments will be described herein in detail, which are represented in the accompanying drawings. When the following description relates to the accompanying drawings, the same numerals in the different accompanying drawings indicate the same or similar elements unless otherwise indicated. The embodiments described in the following embodiments do not represent all embodiments consistent with the invention. Rather, they are only examples of devices and methods consistent with some aspects of this invention as detailed in the appended claims.

The terms “first”, “second”, “third”, “fourth”, etc., if any, in the specification and claims of the present application and the accompanying drawings are used to distinguish similar objects and need not be used to describe a particular order or sequence. It should be understood that the data so used may be interchangeable, where appropriate, so that the embodiments of the present invention described herein can, for example, be practiced in an order other than those illustrated or described herein. In addition, the terms “comprising” and “having”, and any variations thereof, are intended to cover non-exclusive embodiments, e.g., processes, methods, systems, products or devices that comprise a series of steps or units need not be limited to those clearly listed, but may include other steps or units that are not clearly listed or that are inherent to those processes, methods, products or devices.

It should be noted that the user information (including, but not limited to, user device information, user personal information) and data (including, but not limited to, data used for analysis, stored data, displayed data) involved in this invention are authorized by the user or sufficiently authorized by the parties, and the collection, use, and processing of the relevant data comply with the relevant laws, regulations and standards, and provide a corresponding operation portal for users to choose to authorize or refuse.

A nutrient solution is fed to provide subjects such as patients with sufficient nutrients to support their life activities and help their condition recover. Subject information such as weight change, age, and calorie consumption are important factors to focus on in nutrient feeding, and they have a significant impact on the patient's nutrient needs and feeding regimen. In addition, the patient's vital sign status parameter during the infusion process indicates the patient's nutrient tolerance to a certain extent, and only when the vital signs remain stable can the patient tolerate the process of nutrient intake, thus ensuring the effective absorption and utilization of nutrients. Therefore, when determining the parameters of nutrient infusion, the subject information and the vital sign state should be considered comprehensively.

However, at present, nutrient infusion parameters are usually determined by professional nutritionists, who integrate and formulate personalized nutrient feeding programs according to doctor's instructions and the patient's own basic information, vital signs status and other information, in order to provide appropriate nutrient support, and regularly check the performance of the subject's vital signs in the process of nutrient feeding to adjust the parameters in order to provide better nutrient infusion services to patients as much as possible. However, this parameter determination method relies heavily on the experience and ability of the nutritionist, the accuracy of the data is not guaranteed, and it is not applicable to home scenarios. Besides, it is often difficult for the caregiver and family members to set up a reasonable feeding strategy according to the patient's specific situation and vital signs, and the degree of automation and practicability is limited, so that it cannot provide better nutrient infusion for the subject.

In order to solve the above problems, the invention provides a nutrient infusion control method, taking into account that the absorption and utilization of nutrition is strongly related to the information of the subject and the vital signs of the subject. Therefore, in this invention, nutrient infusion parameters are automatically determined directly according to at least one of the nutrient infusion demand information, the information of the subject, and the current parameters of the vital signs status. Through a data analysis model or by comparing the data with a historical feeding strategy, nutrient infusion parameters are automatically determined, and the nutrient infusion parameters are used to control the nutrient infusion device for nutrient infusion. Accordingly, the determination of the nutrient infusion parameters is no longer solely dependent on the nutritionist, the data accuracy is higher, and it is applicable to various scenarios, which is more practical. Furthermore, the invention can also adjust the parameters according to the actual feeding situation during the infusion process, in order to be more suitable for the subject, and to provide a better nutrient infusion for the subject.

The technical solution of the invention and how the technical solution of the invention solves the above technical problems are described in detail with reference to the following specific embodiments, which may be combined with each other, and the same or similar concepts or processes may not be repeated in certain embodiments. Embodiments of the invention will be described below in conjunction with the accompanying drawings.

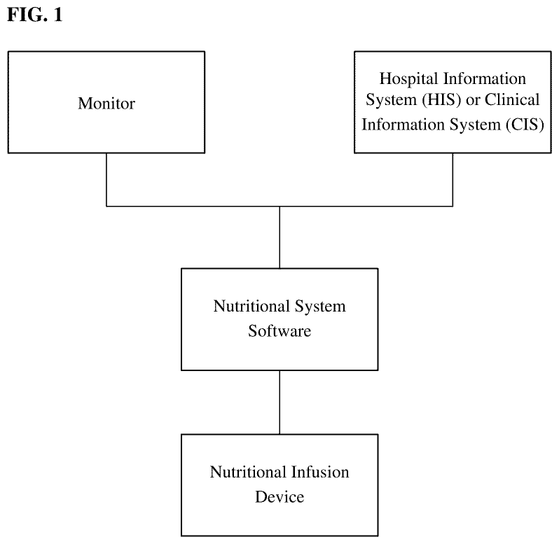

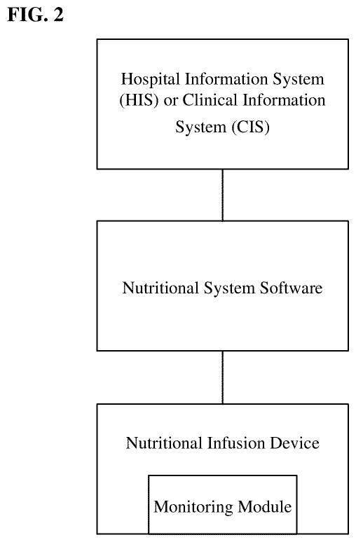

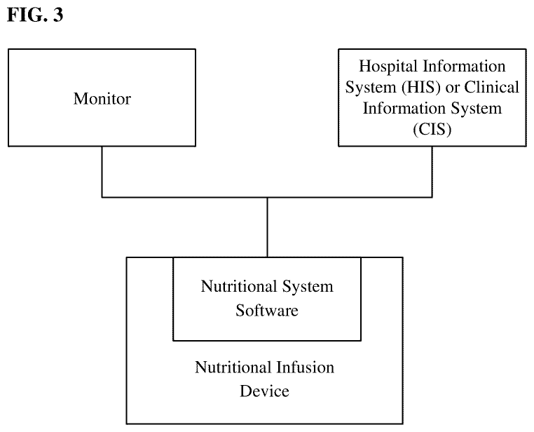

FIG. 1 is a schematic diagram of an application scenario of a nutrient infusion control method provided by embodiments of the invention. As shown in FIG. 1, in this scenario, at least a monitor, a hospital information system (HIS) or a clinical information system (CIS), a nutrient system software, and a nutrient infusion device are included. Both the monitor and the nutrient system software may exist independently of the nutrient infusion device, and the monitor may be connected to the nutrient infusion device by a fixed connection or a pluggable means. FIG. 2 shows a schematic diagram of another application scenario of the nutrient infusion control method provided by embodiments of the invention. As shown in FIG. 2, a monitoring module is deployed on the nutrient infusion device. FIG. 3 is a schematic diagram of yet another application scenario of the nutrient infusion control method provided by embodiments of the invention. As shown in FIG. 3, the nutrient system software is deployed on the nutrient infusion device.

The subject patient's vital sign status parameter may be acquired by the monitor/monitoring module, the subject information may be acquired by the hospital information system or the clinical information system, and the nutrient infusion demand information may be acquired by the nutrient system software. The nutrient system software may determine a first nutrient infusion parameter according to at least one of the nutrient infusion demand information, the subject information, and the vital sign status parameter. The nutrient infusion device may be controlled to perform nutrient infusion according to the first nutrient infusion parameter. It should be noted that the specific structure of the nutrient infusion device is not limited in this invention.

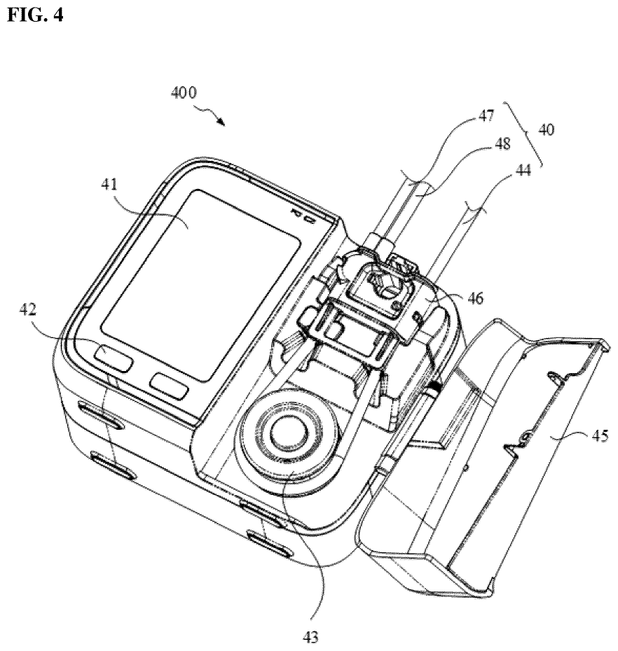

As an example, FIG. 4 shows a structural schematic diagram of a nutrient infusion apparatus provided by embodiments of the invention. As shown in FIG. 4, the nutrient infusion device 400 may include components such as a display 41, one or more buttons 42, a drive actuator 43, a silicone tube 44, a pump door 45, a consumable support plate 46, a nutrient solution line 47, and a flush solution line 48. In addition, the nutrient infusion device 400 includes at least a controller (not shown in the figures), and a drive member (not shown in the figures). The controller is electrically connected to the drive member, the drive member is drivingly connected to the drive actuator 43, and the drive actuator 43 is provided in a piping path of the delivery tube 40 (e.g., silicone tube 44, nutrient fluid line 47, or fluid solution line 48). The drive member, under the control of the controller, drives the drive actuator 43 to rotate so as to drive the fluid flow in the delivery tube 40 for nutrient infusion.

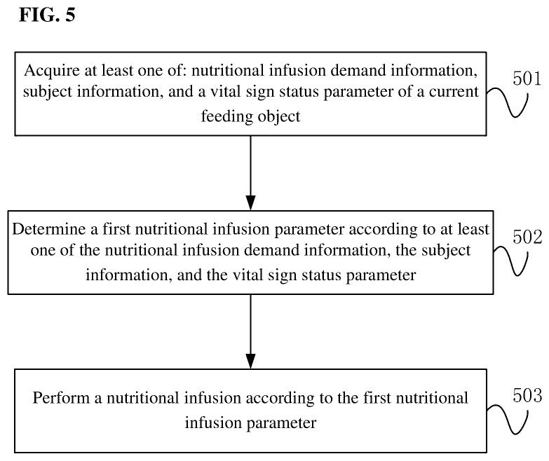

FIG. 5 shows a flowchart of a nutrient infusion control method provided by an embodiment of the invention. The implementation subject of the embodiment of the invention may be a nutrient infusion control device, which may be disposed on a nutrient infusion device or a medical device connected to the nutrient infusion device, such as an infusion central station, an infusion workstation, and an infusion monitoring system, which is not limited in this invention. The embodiments of the invention are described in detail with the implementation subject being the nutrient infusion control device as an example.

As shown in FIG. 5, the embodiment provides a nutrient infusion control method, including the following steps.

S501, at least one of nutrient infusion demand information, subject information, and vital sign status parameter of a current subject are acquired.

As an example, before performing the nutrient infusion, the nutrient infusion control device of the invention first needs to acquire at least one of: the nutrient infusion demand information, the subject information, and the vital sign status parameter of the current subject.

The nutrient infusion demand information indicates the nutrient feeding demand of the healthcare personnel for the subject, which may be input by the healthcare personnel, family members and other personnel according to the practical demand, or may be inputted from other systems. As an example, the nutrient infusion demand information may include one or more of: a nutrient solution type, a feeding mode, a feeding rate, a feeding amount, a feeding time, and the like.

The subject information characterizes basic information about the individual patient, which may be retrieved from a hospital information system HIS or a clinical information system CIS. For example, the subject information may include one or more of: the patient's weight, weight change, age, calorie consumption, height, gender, patient type, and information on the diseases suffered by the patient.

During the nutrient feeding process, changes in the patient's basic personal information need to be closely monitored to determine if nutrient intake is appropriate. For example, if weight loss persists, it may indicate inadequate nutrient intake, requiring an increase in nutrient intake or an adjustment of the nutrient formula. On the contrary, if weight gain persists, there may be a problem of overfeeding, requiring an appropriate reduction in calorie intake. In some examples, there are differences in the nutrient needs and tolerance of patients of different ages. Young children and the elderly have relatively weaker digestive and immune systems and require more carefully selected feeding regimens and nutrient formulas. In some examples, some special diseases such as diabetes mellitus, renal insufficiency and other conditions also require special attention to the dietary composition and nutrient intake of patients. In some examples, the calorie consumption required by a patient depends on his/her physical condition, activity level, and medical condition. If the calorie intake is insufficient, it may lead to malnutrition and decreased immunity, whereas if the calorie intake is too high, it may lead to long-term complications such as obesity and cardiovascular disease. It is evident that information about the feeding goal is crucial for infusion control.

The vital sign status parameter characterizes the actual physical condition of the user, and the stability of vital signs is the basis for nutrient feeding. The process of nutrient intake may, for example, only be tolerated by the patient if the vital signs remain stable, thus ensuring effective absorption and utilization of nutrients. The vital sign status parameter may be acquired by monitoring with devices such as monitors/monitoring modules. For example, the vital sign status parameter may include one or more of: an end-expiratory carbon dioxide indicator EtCO2, a hemodynamic indicator, a blood glucose indicator, a heart rate, a blood pressure, an electrocardiogram, a body temperature, an oxygen saturation, and a pulse. The information including body temperature, heart rate and blood pressure in the vital sign status parameter can reflect the overall health status of the patient, which is conducive to understanding the nutrient needs of the patient and facilitating the adjustment of the amount and type of nutrient intake. The changes in vital signs can also reflect the metabolic status and intestinal function of the patient, e.g., an elevated temperature may indicate infection, and an accelerated heart rate and rapid respiration may indicate hypoxia. Attention to these vital signs status changes can provide patients with a better nutrient infusion plan to ensure the effective absorption and utilization of nutrition.

S502, the first nutrient infusion parameter is determined according to at least one of the nutrient infusion demand information, the subject information, and the vital sign status parameter.

As an example, after the nutrient infusion control device of this invention acquires at least one of the nutrient infusion demand information, the subject information, and the vital sign status parameter of the current subject, the first nutrient infusion parameter may be determined according to the acquired information. The first nutrient infusion parameter refers to parameter information related to the infusion, and may include one or more of: a nutrient solution type, an infusion mode, an infusion rate, an infusion volume, and the like. When the feeding mode is intermittent infusion, the first nutrient infusion parameter may also include information such as a single infusion volume, an infusion time of each time, an infusion rate of each infusion, and an interval time, which is not limited in this invention.

In addition, the invention does not limit how the first nutrient infusion parameter can be determined according to at least one of the nutrient infusion demand information, the subject information, and the vital sign status parameter. In one example, the step of determining the first nutrient infusion parameter according to at least one of the nutrient infusion demand information, the subject information, and the vital sign status parameter may include the following steps.

The at least one of the nutrient infusion demand information, the subject information, and the vital sign status parameter are inputted into a predetermined data analysis model, and an infusion strategy output by the predetermined data analysis model is determined as the first nutrient infusion parameter.

As an example, the predetermined data analysis model may be a data analysis model pre-trained for determining a nutrient infusion parameter according to at least one of: the nutrient infusion demand information, the subject information, and the vital sign status parameter of the subject. After the nutrient infusion demand information, the subject information, and the at least one of the vital sign status parameter are input into the predetermined data analysis model, the predetermined data analysis model will output a recommended infusion strategy after data analysis, and the invention may determine that the infusion strategy output by the predetermined data analysis model is the first nutrient infusion parameter.

In addition to determining the first nutrient infusion parameter using the method of the above-described predetermined data analysis model, in other embodiments, a correspondence between the inputs and outputs may be established by a machine learning algorithm, a neural network algorithm, a clustering algorithm, an expert system, or the like, so as to acquire a more accurate first nutrient infusion parameter by means of the acquired nutrient infusion demand information, the subject information, and the vital sign status parameter.

For example, if the first nutrient infusion parameter is determined according to the nutrient infusion demand information, then after acquiring the nutrient infusion demand information, the type of nutrient solution in the nutrient infusion demand information is determined to be the type of nutrient solution in the first nutrient infusion parameter, the feeding rate is determined to be the infusion rate, the feeding amount is determined to be the infusion amount, and so on. The first nutrient infusion parameter may be determined directly or indirectly according to the parameters related to the nutrient infusion demand information.

If the first nutrient infusion parameter is determined according to the subject information, the age, gender, patient type, and disease information of the patient in the subject information are extracted, and the first nutrient infusion parameter matching the subject information may be selected according to a preset feeding treatment program. If the subject is a child or an elderly person, the type of nutrient solution, the feeding rate and the infusion mode suitable for the child or the elderly may be selected according to the preset feeding treatment program. If the subject is an obese patient, however, a nutrient solution with lower calories and the infusion volume that is less than that of the normal subject may be selected.

In addition, during the process of feeding, the vital sign information of the subject can be acquired by the monitoring device, and the first nutrient infusion parameters can be determined according to the vital sign status information. For example, when the blood glucose value and the rate of change of the blood glucose of the subject are detected, the infusion rate and the infusion volume of the first nutrient infusion parameter can be determined accordingly. Of course, it is also possible to acquire other vital sign information of the subject other than blood glucose to determine the first nutrient infusion parameters; the examples given here are not an exhaustive list.

If the first nutrient infusion parameters are determined according to the nutrient infusion demand information and the subject information, the feeding rate in the nutrient infusion demand information may be determined to be an initial value of the infusion rate. The feeding volume may then be determined to be an initial value of the infusion volume. Thereafter, the infusion rate may be adjusted according to the age of the subject, and the infusion volume may be adjusted according to the calorie consumption of the subject, the body weight, and the change in body weight, and the like. For example, if the subject is elderly, the infusion rate may be adjusted downward. If the subject's calorie consumption is low and the subject is overweight, the infusion volume may be reduced. Similarly, each of the first nutrient infusion parameters is determined as above.

If the first nutrient infusion parameters are determined according to the nutrient infusion demand information, the subject information, and the vital sign status parameter, the initial value of the first nutrient infusion parameters may be determined according to the nutrient infusion demand information, and the initial value may be adjusted according to the subject information. And next, the start time of infusion may be adjusted according to the heart rate, the blood pressure, the electrocardiogram, and the pulse, and the infusion rate may be adjusted according to the end-of-expiration carbon dioxide index, the hemodynamic index, the blood glucose index, the body temperature, and oxygen saturation. For example, if the end-expiratory carbon dioxide index is low and the hemodynamic index is low, the infusion rate may be reduced to ensure that the subject can tolerate the nutrient intake. Similarly, each of the first nutrient infusion parameters is determined as above.

In summary, when analyzing and determining the nutrient infusion parameters, the relationships among the parameters should be considered comprehensively, and the effect of each parameter on the subject should be confirmed, so as to determine a more accurate nutrient infusion parameter, thereby providing the subject with more reasonable nutrition.

In some examples, the step of determining the first nutrient infusion parameter according to at least one of the nutrient infusion demand information, the subject information, and the vital sign status parameter may include the following steps.

Historical feeding parameters of the current subject are retrieved; the nutrient infusion demand information, the subject information, and the vital sign status parameter are compared and matched with the historical feeding parameters, and the historical feeding parameter with the highest degree of matching is determined as the first nutrient infusion parameter.

As an example, the historical feeding parameters of the current subject may be stored in advance in a database, and these historical feeding parameters may be feeding parameters that have been previously infused for the subject. When determining the first nutrient infusion parameter, the nutrient infusion control device of the invention calls up the historical feeding parameters of the current subject, and compares and matches the information acquired in step S501 with the historical feeding parameters retrieved. A matching degree with each historical feeding parameter may be calculated separately, and then the historical feeding parameter with the highest degree of matching is determined as the first nutrient infusion parameter.

When the first nutrient infusion parameter is determined by matching with the historical feeding parameters, if the first nutrient infusion parameter is displayed, the corresponding matching degree, selected times of use, and other index information may also be displayed, so that the user may better assess the applicability and effectiveness of the first nutrient infusion parameter when it is used as a data reference.

As an example, the first nutrient infusion parameter determined in the above manner may be used as a recommended value of the infusion parameter for the decision maker to use as a data reference, and the nutrient infusion device may be controlled to perform nutrient feeding according to the parameter determined by the decision maker after the decision maker has made a decision. Alternatively, the nutrient infusion may be controlled directly according to the first nutrient infusion parameter, and the invention does not impose any limitation.

Thus, in some possible examples, after determining the first nutrient infusion parameter, the nutrient infusion control method of the invention may further include the following steps.

S1, the first nutrient infusion parameter is displayed.

S2, a confirmation operation from the user is received to accept and apply the first nutrient infusion parameter.

As an example, after determining the first nutrient infusion parameter, the first nutrient infusion parameter may also be displayed in a display interface, and the display interface may also have a parameter confirmation control. After the user operates the confirmation control, the first nutrient infusion parameter may be used for nutrient infusion control. In this embodiment, after the nutrient infusion control device determines the first nutrient infusion parameter, the healthcare personnel should decide whether to authorize the confirmation, and the feeding is started only after manually confirming that the first nutrient infusion parameter meets the feeding requirements, so as to ensure the safety of the nutrient infusion process.

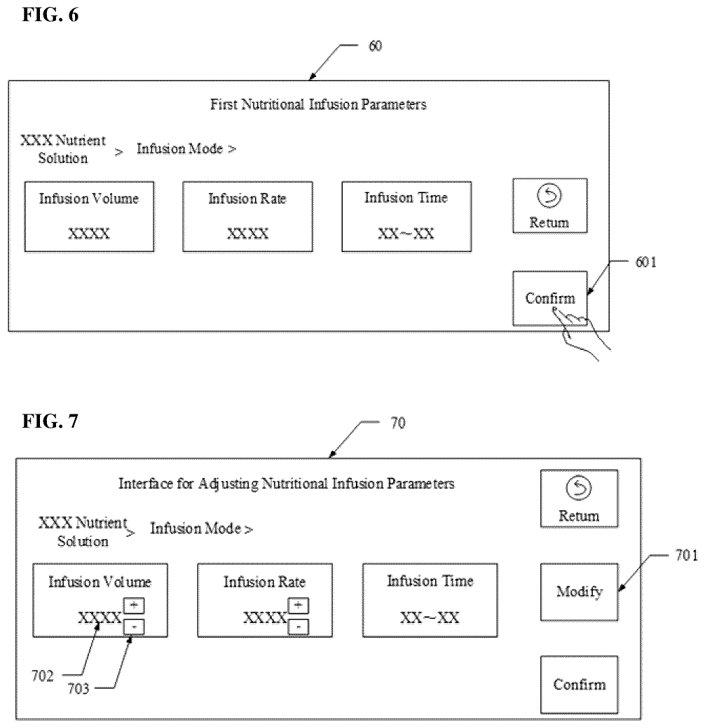

As an example, FIG. 6 shows a schematic diagram of an interface for displaying the nutrient infusion parameters provided by an embodiment of the invention. As shown in FIG. 6, the first nutrient infusion parameters are displayed in the display interface 60: XXX nutrient solution, infusion mode, infusion volume XXXX, infusion rate XXXX, infusion time XX˜XX and other information, and the user may click on the “Confirm” control 601 in the display interface to accept for use the displayed first nutrient infusion parameter.

In some possible examples, when the first nutrient infusion parameters serve as recommended values for the infusion parameters, the user may also adjust the infusion parameter to determine more reasonable infusion parameters. Thus, after determining the first nutrient infusion parameter, the nutrient infusion control method of the invention may further include the following steps.

S10, an interface for adjusting the nutrient infusion parameters is displayed.

S20, the user's input in the interface for adjusting the nutrient infusion parameters is received, and the second nutrient infusion parameter is determined.

S30, the first nutrient infusion parameters are adjusted according to the second nutrient infusion parameter input by the user.

As an example, after determining the first nutrient infusion parameters, an interface for adjusting the nutrient infusion parameters may also be displayed on the display screen. The interface for adjusting the nutrient infusion parameters may display the determined first nutrient infusion parameters, and the user may then input the second nutrient infusion parameter in this interface. The first nutrient infusion parameters may then b adjusted according to the second nutrient infusion parameter input by the user. The second nutrient infusion parameter may be an adjustment value or a target value. When the second nutrient infusion parameter is an adjustment value, the first nutrient infusion parameter is adjusted with the adjustment value indicated by the second nutrient infusion parameter to acquire the new nutrient infusion parameter. When the second nutrient infusion parameter is a target value, the value indicated by the second nutrient infusion parameter is determined to be the new nutrient infusion parameter; the adjusted nutrient infusion parameter is then more consistent with the feeding demand.

As an example, when the user enters the second nutrient infusion parameter in the interface for adjusting the nutrient infusion parameters, the user may directly click on the first nutrient infusion parameter in order to modify it. Alternatively, the user may click on the modification control on this interface for modification, and in the modification mode, the user may directly click on the increase/decrease preset amount button, or, for example, enter the value of the second nutrient infusion parameter.

In some possible examples, an automatic reminder may also be issued to prompt the healthcare worker for parameter adjustment and verification when parameter adjustment is required. The reminder may be in the form of a voice alarm, a flashing alarm light, etc., After the reminder is issued, the interface for adjusting the nutrient infusion parameters may be displayed for the user to make parameter adjustments.

As an example, FIG. 7 shows a schematic diagram of an interface for adjusting the nutrient infusion parameters provided by an embodiment of the invention. In FIG. 7, in the interface 70 for adjusting the nutrient infusion parameters, the first nutrient infusion parameters are shown as: XXX nutrient solution, an infusion mode, an infusion volume XXXX, an infusion rate XXXX, an infusion time XX˜XX, as well as other displayed information. When the user wants to adjust the specific contents of the first nutrient infusion parameters displayed in the interface, the user may, on the basis of the interface 70, directly click on the “Modify” control 701 in the interface to make modifications, or directly click on the parameter itself “XXXX” 702 to enter the corresponding parameter adjustment value, or click the “+”/“−” control to increase/decrease the corresponding value, without limitation of the invention.

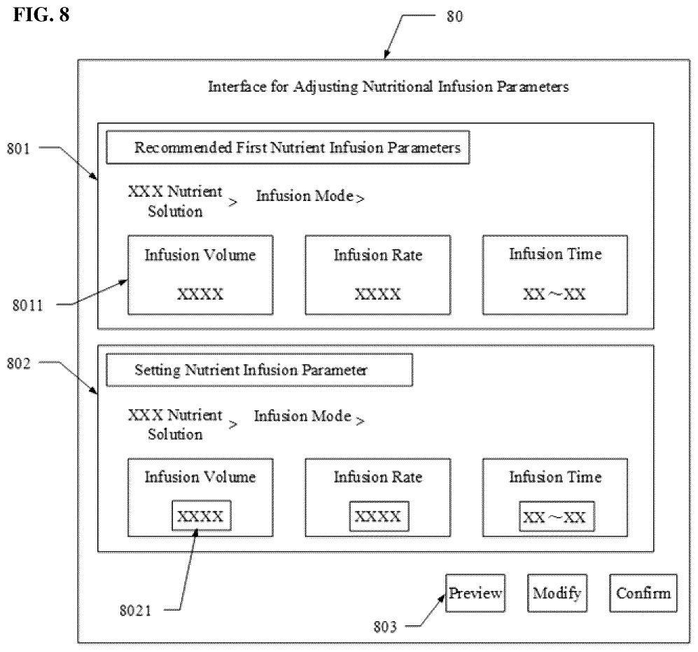

As an example, FIG. 8 shows a schematic diagram of another interface for adjusting nutrient infusion parameters provided by embodiments of the invention. As shown in FIG. 8, the display area 801 in the interface 80 for adjusting the nutrient infusion parameters indicates recommended first nutrient infusion parameters, which are determined according to at least one of: the nutrient infusion demand information, the subject information, and the vital sign status parameter, which may include XXX nutrient solution, the infusion mode, the infusion volume XXXX, the infusion rate XXXX, the infusion time XX˜XX, and other information. The display area 802 is an area for setting the nutrient infusion parameter, and the user can make parameter settings in 802 based on the recommended first nutrient infusion parameters displayed in 801. For example, the user can click “XXXX” 8021 to enter the set infusion amount according to the recommended value of the infusion amount 8011.

When adjusting the nutrient infusion parameter as described above, the adjusted value should be within a permissible range so as to avoid undesirable consequences caused by misadjustment. Thus, in some examples, after receiving the second nutrient infusion parameter inputted by the user in the interface for adjusting the nutrient infusion parameters, the method may further include: determining whether the second nutrient infusion parameter input by the user exceeds a preset threshold value. When it is determined that the second nutrient infusion parameter input by the user exceeds the preset threshold value, a prompting message may be output to prompt the user that the parameter adjustment is out of range, so as to enable the user to correctly adjust the nutrient infusion parameter to ensure that the nutrient infusion parameters are accurate. The prompting information may be a voice alarm, an alarm light flashing, a text prompt, an icon prompt, a highlight prompt, a pop-up window prompt, and the like, and the invention does not impose any limitation.

S503 (FIG. 5), the nutrient infusion is performed according to the first nutrient infusion parameter.

As an example, when the first nutrient infusion parameter is determined, the nutrient infusion control device of the invention controls the nutrient infusion device to perform the nutrient infusion according to the determined first nutrient infusion parameter, thereby completing the feeding of the subject.

The nutrient infusion control method provided by an embodiment of the invention includes: acquiring at least one of nutrient infusion demand information, subject information, and vital sign status parameter of a current subject; determining a first nutrient infusion parameter according to at least one of the nutrient infusion demand information, the subject information, and the vital sign status parameter; and performing a nutrient infusion according to the first nutrient infusion parameter. In the invention, the first nutrient infusion parameter is quickly and automatically determined directly according to at least one of the nutrient infusion demand information, the subject information, and the vital sign status parameter. This approach reduces reliance on nutritionists, enhances data accuracy, and improves the precision of nutrient infusion control. Furthermore, this method is applicable to various practical scenarios, allowing for rapid and accurate determination of the first nutrient infusion parameters even in home environments, and thus controlling the nutrient infusion device with higher practicality.

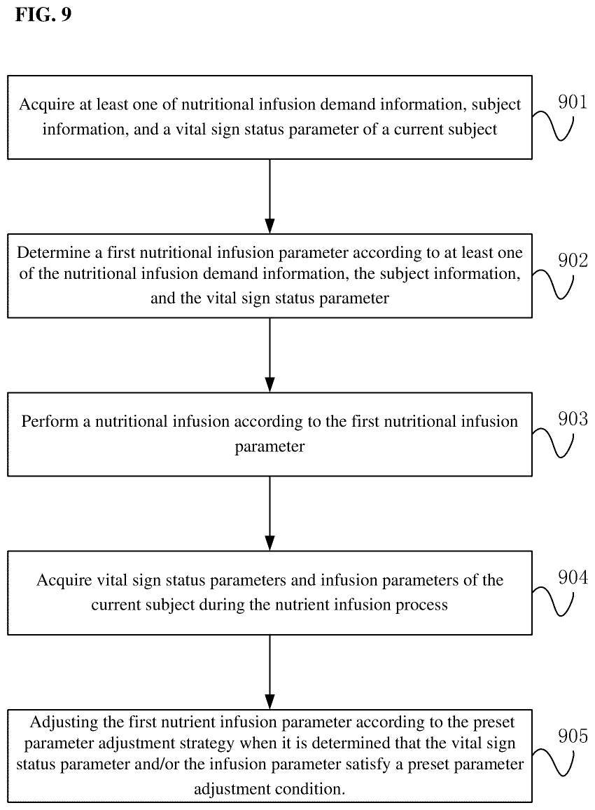

FIG. 9 shows a flowchart of another nutrient infusion control method provided by embodiments of the invention. As shown in FIG. 9, the nutrient infusion control method provided by this embodiment comprises:

S901, at least one of: nutrient infusion demand information, subject information, and vital sign status parameter of the current subject are acquired.

S902, a first nutrient infusion parameter is determined according to at least one of: the nutrient infusion demand information, the subject information, and the vital sign status parameter.

S903, the nutrient infusion is performed according to the first nutrient infusion parameter.

The specific realization of step S901 to step S903 is similar to step S501 to step S503, and will not be repeated here.

S904, vital sign status parameters and infusion parameters of the current subject are acquired during the nutrient infusion process.

As an example, in practice, during the feeding process, the vital signs of the subject will change, and the actual infusion parameters will differ from the first nutrient infusion parameters previously determined, when the changes in these parameters will have a certain effect on the infusion effect. For this reason, in order to improve the infusion effect and ensure effective absorption and utilization of nutrients, the nutrient infusion control device of the invention can also acquire the vital sign status parameter and the infusion parameters of the current subject during the nutrient infusion process, so as to adjust the first nutrient infusion parameters according to the real-time state of the current subject, in order to make the infusion control more accurate.

S905, the first nutrient infusion parameter is adjusted according to the preset parameter adjustment strategy when it is determined that the vital sign status parameter and/or the infusion parameter satisfy a preset parameter adjustment condition.

As an example, when adjusting the first nutrient infusion parameter, the nutrient infusion control device of this invention may determine whether the vital sign status parameter and/or the infusion parameter satisfy the preset parameter adjustment conditions. If it is determined that the vital sign status parameter and/or the infusion parameter satisfy the preset parameter adjustment condition, then the first nutrient infusion parameter is adjusted according to the preset parameter adjustment strategy. The parameter adjustment conditions and the parameter adjustment strategy may be set according to the practical situation, and the invention does not impose any limitation.

The key to controlling the nutrient solution input lies in the precise adjustment of the infusion rate or the pump speed. When the subject cannot tolerate the preset target infusion rate, the practical infusion rate should be reduced to avoid an adverse reaction, and when the general condition of the subject is good, the system can gradually increase the infusion rate until the preset target infusion rate is reached, so as to ensure that the expected feeding goal is accomplished within the expected time.

As an example, the infusion rate may be related to a plurality of parameters, and each factor should be considered comprehensively when adjusting the infusion rate, which may be adjusted using the following adjustment formula:

$${υ}_{2} = F ( {Info}_{subject} , E n {t}_{solution} , EtCO 2 , {I}_{HDN} , BG , Δυ , Δ V ) .$$

υ₂ represents the adjusted infusion rate, Info_{subject} represents subject information (e.g., subject's weight, weight change, age, calorie consumption, etc.), Entₛₒₗᵤₜᵢₒₙ represents the type of nutrient solution, EtCO2 represents the end-expiratory carbon dioxide index, I_{HDN} represents the hemodynamic index, BG represents the blood glucose index, and Δυ represents the difference between the current actual infusion rate and the current preset target infusion rate, and ΔV represents the difference between the current actual infused volume and the current theoretical volume that should be infused. The adjustment formula is an empirical formula derived according to clinical experience, and an optimal adjustment speed υ₂ may be derived by performing a differential analysis according to these parameters.

When adjusting the infusion rate according to the above information, it should be taken into account that the end-expiratory carbon dioxide indicator EtCO2 may reflect the circulatory status of the patient, and a low end-expiratory carbon dioxide indicator EtCO2 may reflect hypocapnia, which has deleterious effects such as low blood pressure due to peripheral vasodilatation, insufficient perfusion of the tissues, cardiac arrhythmia, constriction of the cerebral vasculature and decreased cognitive ability. A faster nutrient feeding rate may exacerbate these symptoms when values of the end-expiratory carbon dioxide indicator EtCO2 are below the preset threshold, and therefore, the feeding rate needs to be lower at this time.

In some examples, the hemodynamic index IHDN includes pressure parameters, flow parameters, and volumetric parameters, etc., the combination of which can reflect the patient's hemodynamic status. When the patient has hypotension, tachycardia or bradycardia and other hemodynamic instability, the gastrointestinal tract may be ischemic or under-perfused. At such time, it is necessary to lower the feeding rate to avoid possible triggering of gastrointestinal intolerance or other complications, in order to reduce the burden on the gastrointestinal tract.

In some examples involving the blood glucose indicator BG, high blood glucose may lead to gastrointestinal dysfunction and affect intestinal absorption and digestion. Therefore, for patients with unstable blood glucose control, a lower feeding rate should preferably be used, especially for nutrient solutions with high sugar content.

In addition, according to the difference Δυ between the current actual infusion rate and the current preset target infusion rate, and the difference ΔV between the current actual infused volume and the current theoretical volume that should be infused, the process speed control adjusted to υ2 can also be determined, controlling the infusion rate to be adjusted in a way that provides a higher “comfort” level, so as to enable the subject to better tolerate and improve tolerance, thereby improving tolerance and infusion “comfort”. For example, if the subject is in good condition but the current feeding volume falls short of expectations (ΔV is negative) and the current infusion rate is lower than the preset target infusion rate (Δυ is negative), the infusion rate can be adjusted slowly towards the target rate υ₂ according to the degree of difference and parameters such as subject information and nutrient type

In addition, for adjustment of other nutrient infusion parameters such as the type of nutrient solution, the infusion mode, and the infusion amount, the adjustment method is similar to the adjustment method of the infusion rate, and the current vital sign status parameter and infusion parameters of the subject should be considered comprehensively, and the current infusion parameters should be adjusted to provide a more suitable nutrient intake for the subject, and to ensure effective absorption and utilization of the nutrients.

In one possible example, after the step of adjusting the first nutrient infusion parameter, in order to facilitate the healthcare personnel to view the adjustment record, conduct an infusion review, and more accurately understand the specifics of the infusion process, the nutrient infusion control method of the invention may further include the following step.

Details of the parameter adjustment during the nutrient infusion process are records. The details of the parameter adjustment include a parameter adjustment time and/or the values before and after the parameter adjustment.

As an example, by recording the parameter adjustment time and the values before and after the parameter adjustment during the nutrient infusion process, the user can be helped to more accurately understand the nutrient intake status of the subject, so as to subsequently propose a more suitable nutrient infusion program. For example, in the interface 80 for adjusting the nutrient infusion parameters shown in FIG. 8, by clicking on the “preview” control 803, the user can view the parameter adjustment details.

The nutrient infusion control method provided by an embodiment of the invention includes: acquiring at least one of nutrient infusion demand information, subject information, and vital sign status parameter of a current subject; determining a first nutrient infusion parameter according to at least one of the nutrient infusion demand information, the subject information, and the vital sign status parameter; performing a nutrient infusion according to the first nutrient infusion parameter; d acquiring a vital sign status parameter and an infusion parameter of the current subject during the nutrient infusion process; and adjusting the first nutrient infusion parameter according to a preset parameter adjustment strategy when it is determined that at least one of the vital sign status parameter and the infusion parameter satisfies a preset parameter adjustment condition. The invention not only quickly and automatically determines the first nutrient infusion parameter directly according to at least one of: the nutrient infusion demand information, the subject information, and the vital sign status parameter, so that the determination of the nutrient infusion parameter no longer depends solely on the nutritionist, and its data accuracy is also higher, so as to improve the accuracy of the control of the nutrient infusion. Also, during the infusion process, the infusion parameters are adjusted in a timely manner according to the actual feeding situation, so as to make it more suitable for the subject, provide a better nutrient infusion for the subject, and ensure the effective absorption and utilization of nutrition.

The following are examples of the apparatus embodiments of this invention, which can be used to perform the method embodiments of the invention. For details not disclosed in the apparatus embodiments of the invention, please refer to the method embodiments of this invention.

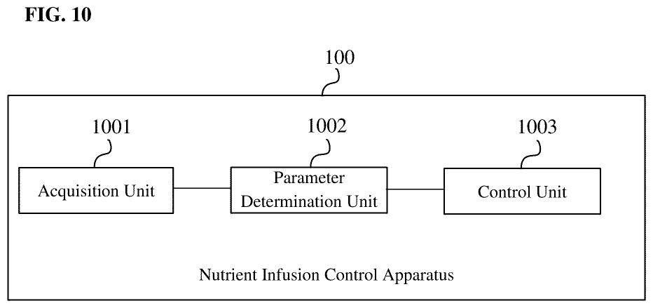

FIG. 10 shows a structural schematic diagram of a nutrient infusion control apparatus provided by embodiments of the invention. As shown in FIG. 10, the nutrient infusion control apparatus 100 provided by the embodiments of the invention includes an acquisition unit 1001, a parameter determination unit 1002, and a control unit 1003.

The acquisition unit 1001 is configured to acquire at least one of nutrient infusion demand information, subject information, and vital sign status parameter of a current subject.

The parameter determination unit 1002 is configured to determine a first nutrient infusion parameter according to at least one of: the nutrient infusion demand information, the subject information, and the vital sign status parameter.

The control unit 1003 is configured to perform the nutrient infusion according to the first nutrient infusion parameter.

The apparatus provided in this embodiment may be used to perform the methods of the above embodiments, the realization principle and technical effect of which are similar and will not be repeated herein.

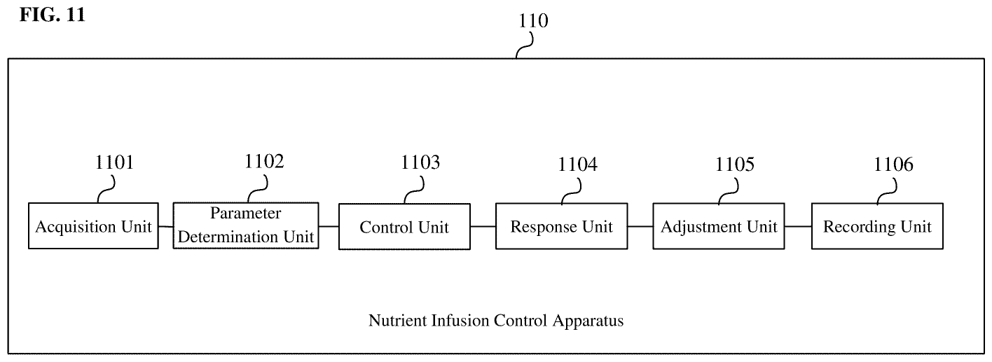

FIG. 11 shows a structural schematic diagram of another nutrient infusion control apparatus provided by an embodiment of the invention. As shown in FIG. 11, the nutrient infusion control apparatus 110 provided by the embodiment of the invention includes an acquisition unit 1101, a parameter determination unit 1102, and a control unit 1103.

The acquisition unit 1101 is configured to acquire at least one of nutrient infusion demand information, subject information, and vital sign status parameter of a current subject.

The parameter determination unit 1102 is configured to determine a first nutrient infusion parameter according to at least one of the nutrient infusion demand information, the subject information, and the vital sign status parameter.

The control unit 1103 is configured to perform the nutrient infusion according to the first nutrient infusion parameter.

In one example, the nutrient infusion demand information includes one or more of the following: a nutrient solution type, a feeding mode, a feeding rate, a feeding amount, and a feeding time;
The subject information includes one or more of the following: weight, weight change, age, calorie consumption;
The vital sign status parameter include one or more of the following: end-expiratory carbon dioxide indicator, hemodynamic indicator, blood glucose indicator, heart rate, blood pressure, electrocardiogram, body temperature, blood oxygen saturation, pulse;

The first nutrient infusion parameter includes one or more of the following: type of nutrient solution, infusion mode, infusion rate, infusion volume.

In one example, the apparatus 110 further includes a response unit 1104 configured to display an interface for adjusting nutrient infusion parameters, receive user input in the interface for adjusting the nutrient infusion parameters to determine a second nutrient infusion parameter, and adjust the first nutrient infusion parameter according to the second nutrient infusion parameter input by the user.

In one example, the response unit 1104 is configured to display the first nutrient infusion parameter and to receive a confirmation operation from the user to adopt the first nutrient infusion parameter.

In one example, the parameter determining unit 1102, may be configured to input at least one of: the nutrient infusion demand information, the subject information, and the vital sign status parameter into a predetermined data analysis model, and determine an infusion strategy output by the predetermined data analysis model as the first nutrient infusion parameter.

In one example, the parameter determining unit 1102 may be configured to retrieve historical feeding parameters of the current subject, compare and match the nutrient infusion demand information, the subject information, and the vital sign status parameter with the historical feeding parameters, and determine the historical feeding parameter with the highest degree of matching as the first nutrient infusion parameter.

In one example, the acquisition unit 1101 is further configured to acquire the vital sign status parameter and the infusion parameter of the current subject during the nutrient infusion process. The apparatus further includes an adjustment unit 1105, which is configured to adjust the first nutrient infusion parameter according to a preset parameter adjustment strategy when it is determined that at least one of the vital sign status parameter and the infusion parameter satisfies a preset parameter adjustment condition.

In one example, the apparatus further includes a recording unit 1106, which is configured to record details of parameter adjustment during the nutrient infusion process. The details of parameter adjustment include at least one of: a parameter adjustment time and values before and after the parameter adjustment.

This embodiment provides an apparatus that can be configured to perform the method of the above embodiment, the realization principle and technical effect of which are similar and will not be repeated herein.

It should be understood that the division of the various modules of the above apparatus is merely a logical functional division, and may be fully or partially integrated into a physical entity or physically separated when actually realized. These modules can all be realized in the form of software invoked through processing elements; may also be realized in the form of hardware; may also be part of the module through the processing elements invoked in the form of software, part of the module through the form of hardware. In addition, it may also be stored in the form of program code in the memory of the above apparatus, which is called by one of the processing elements of the above apparatus and performs the functions of the above data processing module. Other modules are realized similarly. In addition, all or some of these modules may be integrated together or may be realized independently. The processing element herein may be an integrated circuit with signal processing capability. In the realization, the steps of the above method or each of the above modules may be accomplished by integrated logic circuits of hardware in the processor element or by instructions in the form of software.

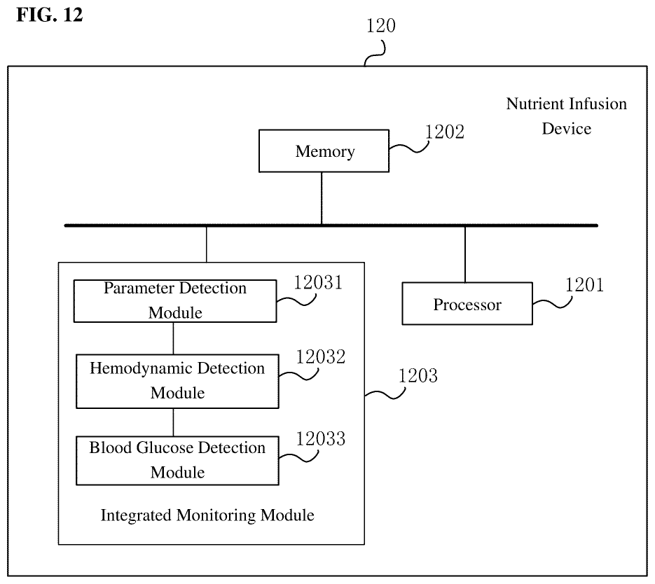

FIG. 12 shows a structural schematic diagram of a nutrient infusion device provided by an embodiment of the invention. As shown in FIG. 12, the nutrient infusion device 120 includes a processor 1201, and a memory 1202 communicatively connected to the processor. Furthermore, an integrated monitoring module 1203 is provided on the nutrient infusion device 120, and the integrated monitoring module is configured to collect the vital sign status parameter of a subject.

The memory 1202 is configured to store computer-executable instructions; and the processor 1201 is configured to execute the computer-executable instructions stored in the memory 1202 to implement the method as in any of the preceding embodiments.

In one example, the integrated monitoring module 1203 includes one or more of the following modules: a respiratory parameter detection module 12031, a hemodynamic detection module 12032, and a blood glucose detection module 12033. The respiratory parameter detection module 12031 is configured to collect end-expiratory carbon dioxide indicators, the hemodynamic detection module 12032 is configured to collect hemodynamic indicators, and the blood glucose detection module 12033 is configured to collect blood glucose indicators.

In one example, the integrated monitoring module is set to be pluggable.

In the specific implementation of the above-described nutrient infusion device, it should be understood that the processor may be a Central Processing Unit (CPU), and may be other general purpose processors, a Digital Signal Processors (DSP), an Application Specific Integrated Circuit (ASIC), a Digital Signal Processor (DSP), and a Specialized Integrated Circuit (ASIC). The general-purpose processor may be a microprocessor or the processor may also be any conventional processor. The method disclosed in conjunction with embodiments of the invention may be directly embodied in a hardware processor to perform the completion, or performed with a combination of hardware and software modules in the processor to perform the completion.

Embodiments of the invention further provide a computer-readable storage medium having computer-executable instructions stored therein. The computer-executable instructions are configured to realize the method as in any of the foregoing when executed by a processor.

It will be appreciated by those of ordinary skill in the art that all or some of the steps for implementing each of the foregoing method embodiments may be accomplished by hardware associated with the computer instructions. The foregoing instructions may be stored in the computer-readable storage medium. The instructions, when executed, performs the steps including each of the above-described method embodiments. The foregoing storage medium may be a ROM, a RAM, a magnetic disk, or a CD-ROM, and other medium that can store program code.

Embodiments of the invention further provide a computer program product including a computer program which, when executed by a processor, is configured to implement a method as in any of the foregoing.

Other embodiments of this invention will readily come to mind to those skilled in the art upon consideration of the specification and practice of the invention disclosed herein. The invention is intended to cover any variations, uses, or adaptations of the invention that follow the general principles of the invention and include common knowledge or customary technical means in the art not disclosed herein. The specification and embodiments are to be regarded as exemplary only, and the true scope and spirit of this invention is indicated by the following claims.

It is to be understood that this invention is not limited to the precise structure which has been described above and illustrated in the accompanying drawings, and that various modifications and changes may be made without departing from its scope. The scope of this invention is limited only by the appended claims.

1. A nutrient infusion control method, comprising:
acquiring at least one of: nutrient infusion demand information, subject information, and a vital sign status parameter of a current subject;
determining a first nutrient infusion parameter as at least one of the nutrient infusion demand information, the subject information, and the vital sign status parameter; and
performing a nutrient infusion according to the first nutrient infusion parameter.

2. The method of claim 1, wherein the nutrient infusion demand information includes at least one of the following: a nutrient solution type, a feeding mode, a feeding rate, a feeding amount, and a feeding time;
the subject information includes at least one of the following: weight, weight change, age, caloric consumption;
the vital sign status parameter includes at least one of the following: an end-expiratory carbon dioxide indicator, a hemodynamic indicator, a blood glucose indicator, a heart rate, a blood pressure, an electrocardiogram, a body temperature, a blood oxygen saturation, a pulse; and
the first nutrient infusion parameter includes at least one of the following: a type of nutrient solution, an infusion mode, an infusion rate, an infusion volume.

3. The method of claim 1, further comprising:
displaying an interface for adjusting at least the first nutrient infusion parameter;
receiving input from the user in the interface for adjusting at least the first nutrient infusion parameter to determine a second nutrient infusion parameter;
adjusting at least the first nutrient infusion parameter according to a second nutrient infusion parameter input by the user.

4. The method of claim 1, wherein, before the step of performing the nutrient infusion according to at least the first nutrient infusion parameter, the method further comprises:
displaying at least the first nutrient infusion parameter;
receiving a confirmation operation from the user to accept and apply at least the first nutrient infusion parameter.

5. The method of claim 1, wherein the step of determining the first nutrient infusion parameter as at least one of the nutrient infusion demand information, the subject information, and the vital sign status parameter, comprises:
inputting at least one of: the nutrient infusion demand information, the subject information, and the vital sign status parameter into a predetermined data analysis model, and determining an infusion strategy output from the predetermined data analysis model as the first nutrient infusion parameter.

6. The method of claim 1, wherein the step of determining the first nutrient infusion parameter as at least one of the nutrient infusion demand information, the subject information, and the vital sign status parameter comprises:
retrieving historical feeding parameters of the current subject;
comparing and matching the nutrient infusion demand information, the subject information, and the vital sign status parameter with the historical feeding parameters; and
determining the historical feeding parameter with the highest degree of matching as the first nutrient infusion parameter.

7. The method of claim 1, further comprising:
acquiring the vital sign status parameter and an infusion parameter of the current subject during the nutrient infusion process; and
adjusting the first nutrient infusion parameter according to a preset parameter adjustment strategy when it is determined that at least one of the vital sign status parameter and the infusion parameter satisfies a preset parameter adjustment condition.

8. The method of claim 7, further comprising:
recording details of parameter adjustment during the nutrient infusion process; wherein the details of parameter adjustment comprise at least one of a parameter adjustment time and values before and after parameter adjustment.

9. A nutrient infusion control apparatus, comprising:
an acquisition unit configured to acquire at least one of: nutrient infusion demand information, subject information, and vital sign status parameter of a current subject;
a parameter determination unit configured to determine a first nutrient infusion parameter according to at least one of the nutrient infusion demand information, the subject information, and the vital sign status parameter; and
a control unit configured to perform a nutrient infusion according to the first nutrient infusion parameter.

10. A nutrient infusion device, provided with an integrated monitoring module; the integrated monitoring module being configured to collect vital sign status parameter of the subject;
wherein the nutrient infusion device further comprises a processor and a memory communicatively connected to the processor;
the memory stores computer-executable instructions;
the processor is configured to execute the computer-executable instructions stored in the memory to:
acquire at least one of nutrient infusion demand information, subject information, and vital sign status parameter of a current subject;
determine a first nutrient infusion parameter according to at least one of the nutrient infusion demand information, the subject information, and the vital sign status parameter; and
perform a nutrient infusion according to the first nutrient infusion parameter . . . .

11. The nutrient infusion device of claim 10, wherein the integrated monitoring module comprises one or more of the following modules: a respiratory parameter detection module, a hemodynamic detection module, and a blood glucose detection module;
wherein the respiratory parameter detection module is configured to collect end-expiratory carbon dioxide indicators, the hemodynamic detection module is configured to collect hemodynamic indicators, and the blood glucose detection module is configured to collect blood glucose indicators.

12. The nutrient infusion device of claim 10, wherein the processor is further configured to:
display an interface for adjusting nutrient infusion parameters;
receive input from the user in the interface for adjusting nutrient infusion parameters to determine a second nutrient infusion parameter; and
adjust the first nutrient infusion parameter according to the second nutrient infusion parameter input by the user.

13. The nutrient infusion device of claim 10, wherein, before the step of performing the nutrient infusion according to the first nutrient infusion parameter, the processor is further configured to:
display the first nutrient infusion parameter;
receive a confirmation operation from the user to adopt the first nutrient infusion parameter.

14. The nutrient infusion device of claim 10, wherein the processor determines the first nutrient infusion parameter according to at least one of: the nutrient infusion demand information, the subject information, and the vital sign status parameter by:
a predetermined data analysis model that inputs at least one of the nutrient infusion demand information, the subject information, and the vital sign status parameter, and determines an infusion output from the predetermined data analysis model as the first nutrient infusion parameter.

15. The nutrient infusion device of claim 10, wherein the processor determines the first nutrient infusion parameter according to at least one of the nutrient infusion demand information, the subject information, and the vital sign status parameter by:
retrieving historical feeding parameters of the current subject;
comparing and matching the nutrient infusion demand information, the subject information, and the vital sign status parameter with the historical feeding parameters; and
determining the historical feeding parameter with the highest degree of matching as the first nutrient infusion parameter.

16. The nutrient infusion device of claim 10, wherein the processor is further configured to:
acquire a vital sign status parameter and an infusion parameter of the current subject during the nutrient infusion process; and
adjust the first nutrient infusion parameter according to a preset parameter adjustment strategy when it is determined that at least one of the vital sign status parameter and the infusion parameter satisfies a preset parameter adjustment condition.

17. The nutrient infusion device of claim 16, wherein the processor is further configured to record details of parameter adjustment during the nutrient infusion process; wherein the details of parameter adjustment comprise at least one of a parameter adjustment time and values before and after the parameter adjustment.
